(19) United States
(12) Patent Application Publication
(10) Pub. No.: US 2020/0253773 A1
(43) Pub. Date: Aug. 13, 2020

(54) SYSTEMS AND METHODS FOR SUPPORTING A LEG

(71) Applicant: Gait Dynamics LLC, Baltimore, MD (US)

(72) Inventor: John LOGUE, Baltimore, MD (US)

(73) Assignee: Gait Dynamics LLC, Baltimore, MD (US)

(21) Appl. No.: 16/784,579

(22) Filed: Feb. 7, 2020

Related U.S. Application Data
(60) Provisional application No. 62/802,925, filed on Feb. 8, 2019.

(51) Int. Cl. A61F 5/01

(52) U.S. Cl. CPC A61F 5/0127; A61F 2005/0179

(57) ABSTRACT

Systems and methods for supporting a user's limb are provided. In some embodiments, a system may include a foot plate configured to engage the user's foot, a coupling configured to engage a portion of the user's leg, a first leaf spring disposed between the foot plate and the coupling, and a second leaf spring extending alongside at least a portion of the first leaf spring. In some embodiments, a space may be defined between the first leaf spring and the second leaf spring. In some embodiments, the space between the first leaf spring and the second leaf spring may be configured to receive one or more spacers of a plurality of spacers.

### RELATED APPLICATIONS

This application claims priority to U.S. Provisional Application No. 62/802,925, filed Feb. 8, 2019.

### FIELD OF THE DISCLOSURE

This disclosure relates to systems such as flexible orthoses which can be used to provide support to a patient's leg during, for example, physical rehabilitation.

### BACKGROUND

Individuals suffering from injuries or other clinical conditions may benefit from supportive orthoses. Ankle-foot orthotics (AFO's) are commonly used to provide support to users who wish to retain mobility, but need additional support to do so safely and without risking injury or delaying recovery. AFO's may include a leaf spring, which provides resistance to certain types of motion, including dorsiflexion (or flexion of the foot upward toward the lower leg).

A typical leaf spring can be understood as having a single spring constant or stiffness, which is determined by the geometry and material properties of the leaf spring. This is an important property of the AFO, as it will affect the amount of support provided, as well as the degree of flexibility and mobility permitted to the user while he or she wears the AFO.

Often, however, users would benefit from alterations to the stiffness of the leaf spring after that value has already been set. For example, clinical prediction of the user's needs may not be accurate, and the user may decide that they need more or less support after using the AFO for a period of time. The user's condition may also change after fitting due to progress of disease or progress of training. In other cases, a user's circumstances may change. For example they may move from a home with no stairs to a home with many stairs. Since climbing stairs requires deflecting the leaf spring prior to applying weight bearing load, a stiffer leaf spring may impede stair climbing whereas a more flexible spring would accommodate stair climbing better. Another example is when a patient gains increased ambulatory capacity, they may gain an increased sense of what is possible. As a user gains skill with the orthosis and as they improve their general physical conditioning, they may desire to increase the power available from the orthosis to support new, more demanding activities. These types of customizations are not available using traditional orthoses.

Moreover, a leaf spring having a single spring constant may not be optimal for users who wish to have lower resistance to dorsiflexion for smaller movements (e.g., lifting the foot a small amount to clear the edge of a stair) but higher resistance to dorsiflexion in the context of larger movements (e.g., overextension). Such resistance profiles are not available using traditional orthoses.

Accordingly, there is a need for a system that provides variable stiffness which can be tuned to a patient's needs in accordance with his or her clinical condition, environment, or the activities in which he or she is engaged. Moreover, there is a need for a system which can provide different levels of resistance in response to different degrees of dorsiflexion.

### SUMMARY

The following presents a simplified summary in order to provide a basic understanding of some aspects described herein. This summary is not an extensive overview of the claimed subject matter. It is intended to neither identify key or critical elements of the claimed subject matter nor delineate the scope thereof. Its sole purpose is to present some concepts in a simplified form as a prelude to the more detailed description that is presented later.

In some embodiments, a system for supporting a user's limb may be provided. The system may include one or more of a foot plate configured to engage the user's foot, a coupling configured to engage a portion of the user's leg, a first leaf spring disposed between the foot plate and the coupling, and a second leaf spring extending alongside at least a portion of the first leaf spring. In some embodiments, a space is defined between the first leaf spring and the second leaf spring. In some embodiments, the space between the first leaf spring and the second leaf spring may be configured to receive one or more spacers of a plurality of spacers, such that a dorsiflexion resistance profile of the system differs depending on how many and which of the one or more spacers is positioned within the space.

The system may be configured such that the space between the first leaf spring and the second leaf spring has a lateral distance d₁ when the system is in a first state in which an external load is not applied. The system may be further configured such that the space between the first leaf spring and the second leaf spring has a lateral distance d₂, which is less than d₁, when the system is in a second state in which an external load is applied.

In some embodiments, the distance between the first leaf spring and the second leaf spring may progressively decrease under increasing loads, and a slope of a stress-strain curve of the system may increase when the distance reaches zero. In some embodiments, a spacer may be disposed in the space between the first leaf spring and the second leaf spring.

In some embodiments, the system may include a plurality of spacers including at least a first spacer and a second spacer. The system may have a first dorsiflexion resistance profile when only the first spacer is disposed in the space between the first leaf spring and the second leaf spring. The system may have a second dorsiflexion resistance profile when the second spacer is disposed, alone or in combination with the first spacer, in the space between the leaf spring and the second leaf spring. The second dorsiflexion resistance profile may be different than the first dorsiflexion resistance profile.

In some embodiments, the first spacer may have a first compression characteristic, and the second spacer has a second compression characteristic that is different than the first spacer. In some embodiments, the first spacer may be removable from the space between the first leaf spring and the second leaf spring by applying a load to the system in a plantarflexion direction. In some embodiments, the first spacer may have a first compressing state in which the spacer has a first resistance to compression, and a second compressing state in which the first spacer has a second resistance to compression that is greater than the first resistance to compression. In some embodiments, the first spacer may comprise a foam.

In some embodiments, the system may have a first loading state in which the system has a first resistance to dorsiflexion, and a second loading state in which the system has a second resistance to dorsiflexion that is greater than the first resistance to dorsiflexion. In some embodiments, the system may transition from the first loading state to the second loading state when a predetermined dorsiflexion value is exceeded. In some embodiments, the predetermined dorsiflexion value may be between 6° and 15°.

In some embodiments, a method for supporting a user's limb may be provided. The method may be performed using a system including one or more of: (i) a foot plate configured to engage the user's foot, (ii) a coupling configured to engage a portion of the user's leg, (iii) a first leaf spring disposed between the foot plate and the coupling, and/or (iv) a second leaf spring extending alongside at least a portion of the first leaf spring, such that a space is defined between the first leaf spring and the second leaf spring. In some embodiments, the method may include determining, based on a first use scenario, that a first dorsiflexion resistance profile of the system is desired. The method may further include determining, based on the first dorsiflexion resistance profile, that a first spacer should be placed in the space defined between the first leaf spring and the second leaf spring. In some embodiments, the method may further include placing the first spacer in the space defined between the first leaf spring and the second leaf spring to achieve the first dorsiflexion resistance profile.

In some embodiments, a first spacer may be selected, based on the first dorsiflexion resistance profile, from a plurality of spacers having different compression characteristics. In some embodiments, it may be determined, based on a second use scenario, that a second dorsiflexion resistance profile is desired, and a second spacer having different compression characteristics may be selected. In some embodiments, the second spacer may be placed in the space defined between the first leaf spring and the second leaf spring. In some embodiments, the first spacer may be removed from the space defined between the first leaf spring and the second leaf spring. In some embodiments, the step of removing the first spacer from the space defined between the first leaf spring and the second leaf spring may include applying a load to the system in a plantarflexion direction.

In some embodiments, the first spacer may be a resilient member. In some embodiments, the first spacer may have a first compressing state in which the spacer has a first resistance to compression, and a second compressing state in which the first spacer has a second resistance to compression that is greater than the first resistance to compression. In some embodiments, the first spacer may comprise a foam.

In some embodiments, a plurality of spacers may be placed in the space between the first leaf spring and the second leaf spring.

In some embodiments, the first dorsiflexion resistance profile may include a first loading state in which the system has a first resistance to dorsiflexion, and a second loading state in which the system has a second resistance to dorsiflexion that is greater than the first resistance to dorsiflexion. In some embodiments, the first dorsiflexion resistance profile may include a transition from the first loading state to the second loading state at a predetermined dorsiflexion value. In some embodiments, the predetermined dorsiflexion value may be between 6° and 15°.

Further variations encompassed within the systems and methods are described in the detailed description of the invention below.

### BRIEF DESCRIPTION OF THE DRAWINGS

The accompanying drawings, which are incorporated herein and form part of the specification, illustrate various, non-limiting embodiments of the present invention. In the drawings, like reference numbers indicate identical or functionally similar elements.

FIG. 1 depicts an exemplary system 100 which can be used to support a patient's limb.

FIG. 2 depicts an exploded diagram of certain components of the exemplary system 100.

FIG. 3 shows a side view of the exemplary system 100.

FIGS. 4A and 4B illustrate an exemplary spacing between a first leaf spring 108 and a second leaf spring 110.

FIGS. 5A-5D provide exemplary illustrations of a system which permits variable dorsiflexion resistance profiles.

FIG. 6 depicts an exemplary method 600 for supporting a user's limb.

FIGS. 7A and 7B illustrate an exemplary multi-spacer embodiment.

### DETAILED DESCRIPTION

While aspects of the subject matter of the present disclosure may be embodied in a variety of forms, the following description and accompanying drawings are merely intended to disclose some of these forms as specific examples of the subject matter. Accordingly, the subject matter of this disclosure is not intended to be limited to the forms or embodiments so described and illustrated.

Unless defined otherwise, all terms of art, notations and other technical terms or terminology used herein have the same meaning as is commonly understood by one of ordinary skill in the art to which this disclosure belongs. All patents, applications, published applications and other publications referred to herein are incorporated by reference in their entirety. If a definition set forth in this section is contrary to or otherwise inconsistent with a definition set forth in the patents, applications, published applications, and other publications that are herein incorporated by reference, the definition set forth in this section prevails over the definition that is incorporated herein by reference.

Unless otherwise indicated or the context suggests otherwise, as used herein, “a” or “an” means “at least one” or “one or more.”

This description may use relative spatial and/or orientation terms in describing the position and/or orientation of a component, apparatus, location, feature, or a portion thereof. Unless specifically stated, or otherwise dictated by the context of the description, such terms, including, without limitation, top, bottom, above, below, under, on top of, upper, lower, left of, right of, in front of, behind, next to, adjacent, between, horizontal, vertical, diagonal, longitudinal, transverse, radial, axial, etc., are used for convenience in referring to such component, apparatus, location, feature, or a portion thereof in the drawings and are not intended to be limiting.

Furthermore, unless otherwise stated, any specific dimensions mentioned in this description are merely representative of an exemplary implementation of a device embodying aspects of the disclosure and are not intended to be limiting.

As used herein, the terms “substantially” and “substantial” refer to a considerable degree or extent. When used in conjunction with, for example, an event, circumstance, characteristic, or property, the terms can refer to instances in which the event, circumstance, characteristic, or property occurs precisely as well as instances in which the event, circumstance, characteristic, or property occurs to a close approximation, such as accounting for typical tolerance levels or variability of the embodiments described herein.

As used herein, the terms “optional” and “optionally” mean that the subsequently described, component, structure, element, event, circumstance, characteristic, property, etc. may or may not be included or occur and that the description includes instances where the component, structure, element, event, circumstance, characteristic, property, etc. is included or occurs and instances in which it is not or does not.

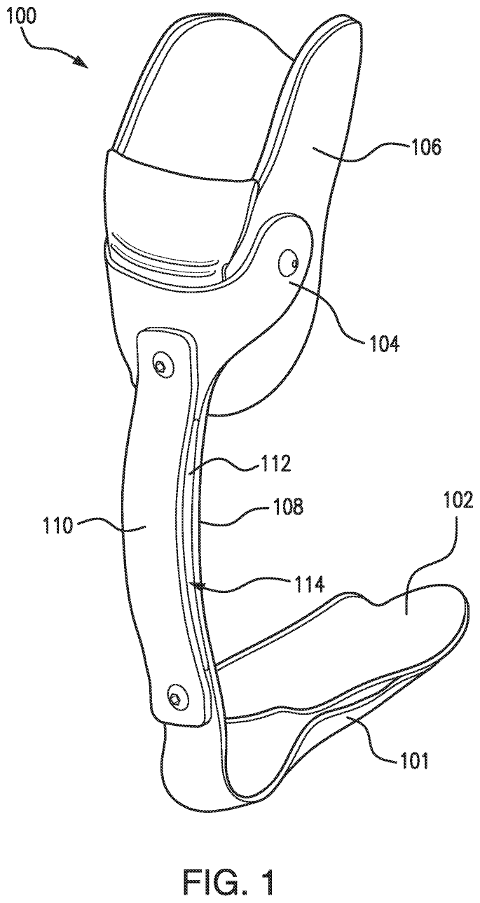

FIG. 1 depicts an exemplary system 100 which can be used to support a patient's limb. In some embodiments, the system may include one or more of a foot plate 101, a coupling 104, a first leaf spring 108, and a second leaf spring 110. In some embodiments, the foot plate 101, coupling 104, and first leaf spring 108 may be formed as a single, unitary piece. For example, the foot plate 101, coupling 104, and first leaf spring 108 may be formed using a pre-preg carbon fiber material. In some embodiments, the second leaf spring may also be formed using a pre-preg carbon fiber material. Other suitable materials may also be used.

In some embodiments, the foot plate 101 may receive an insert 102 which may be molded or padded to comfortably support a user's foot. In some embodiments, the coupling may be configured to engage, directly or indirectly, a portion of the user's leg. For example, as illustrated in the exemplary embodiment shown in FIG. 1, the coupling 104 may engage a cuff 106, which may partially or fully enclose a portion of a user's leg. In some embodiments, the cuff 106 may be configured to engage a user's knee, or the portions of the user's leg above and/or below the knee. The cuff 106 may be padded or molded to comfortably engage the portion of the user's leg that it is configured to contact.

In some embodiments, the second leaf spring 110 may extend alongside at least a portion of the first leaf spring 108. For example, as illustrated in the exemplary embodiment shown in FIG. 1, the second leaf spring 110 may be attached to the first leaf spring 108 at a first position proximate to the coupling 104 and at a second position proximate to the foot plate 101. In some embodiments, this attachment may be made by bolts, rivets, or other hardware. In some embodiments, a space 114 may be defined between the first leaf spring 108 and the second leaf spring 110. In some embodiments, an optional spacer 112 may be disposed in the space between the first leaf spring 108 and the second leaf spring 110. In some embodiments, the spacer 112 may be a resilient member. In some embodiments, the spacer 112 may extend from the first leaf spring 108 to the second leaf spring 110. For example, the spacer 112 may span an entire lateral distance of the space 114. In other embodiments, the spacer 112 may extend only partially across the lateral distance of the space 114. In some embodiments, a plurality of spacers 112 may be disposed in the space 114.

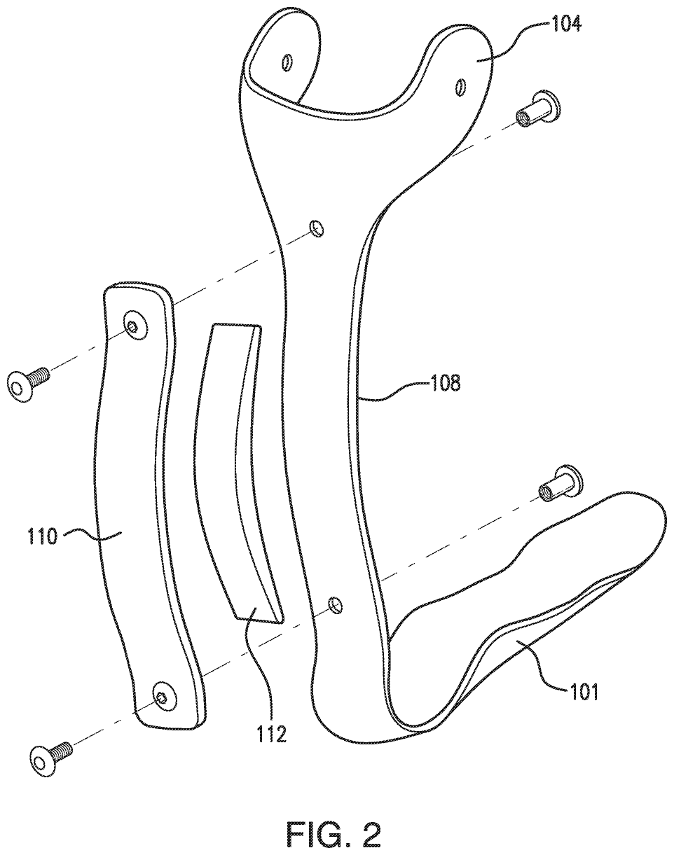

FIG. 2 depicts an exploded diagram of certain components of the exemplary system 100 shown in FIG. 1. As illustrated in FIG. 2, the foot plate 101, coupling 104, and first leaf spring 108 may be formed as a single, unitary structure. The second leaf spring 110 may be attached to the first leaf spring 108 such that it extends alongside the first leaf spring 108 and defines a space therebetween, in which a spacer 112 may be disposed. FIG. 2 illustrates an option for connecting the first leaf spring 108 and second leaf spring 110 by way of hardware extending through holes in both pieces. In some embodiments, the connection may be detachable, to facilitate removal of a spacer 112 and, optionally, replacement with a new spacer 112 having different compression characteristics. In some embodiments, applying a load to the system 100 in a plantarflexion direction may cause the space between the two leaf springs to increase rather that decrease. In some embodiments, applying a load in a plantarflexion direction may allow spacers to be inserted or removed without the need of tools. In some embodiments, a hook and loop fastener may be provided to ensure that the spacers will not inadvertently fall out during plantar flexion.

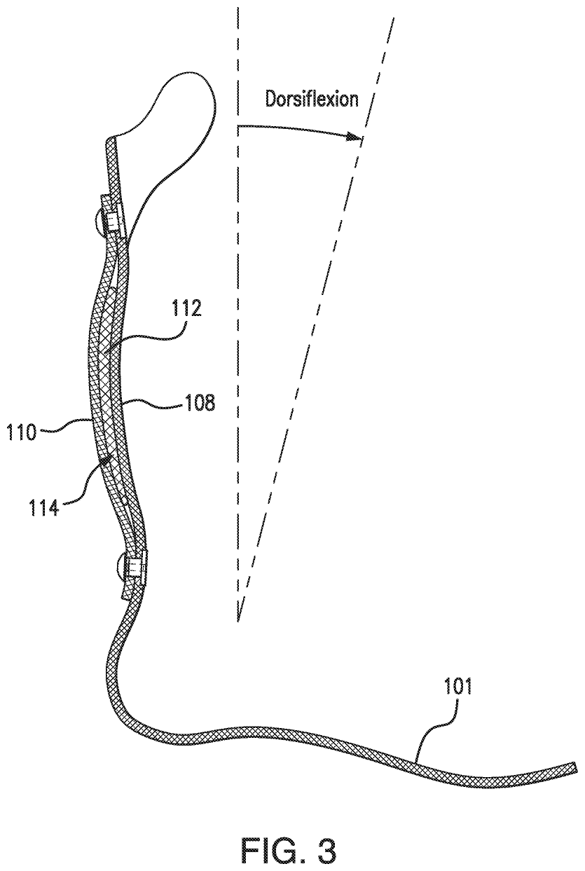

FIG. 3 shows a side view of the exemplary system 100. In some embodiments, the system 100 may be configured to deform under an external load. For example, in a normal gait, an angle between the user's foot and lower leg varies. As a user wearing the system 100 walks (or runs, walks up stairs, etc.), a load may be applied to the system 100 which causes the system to deform. As illustrated in FIG. 3, dorsiflexion refers to bending in a direction that reduces the angle between the foot and the lower leg. In some embodiments, the system 100 may be configured to provide resistance to dorsiflexion, where the amount of resistive force varies depending on the degree of dorsiflexion at a given time. The relationship between degree of dorsiflexion and resistive force may be referred to as a dorsiflexion resistance profile for that system. In some embodiments, a system 100 may have differing dorsiflexion resistance profiles, depending on whether the second leaf spring 110 is attached to the first leaf spring 108, whether one or more spacers 112 are disposed between the first leaf spring 108 and the second leaf spring 110, and the compression characteristics of any such spacers 112.

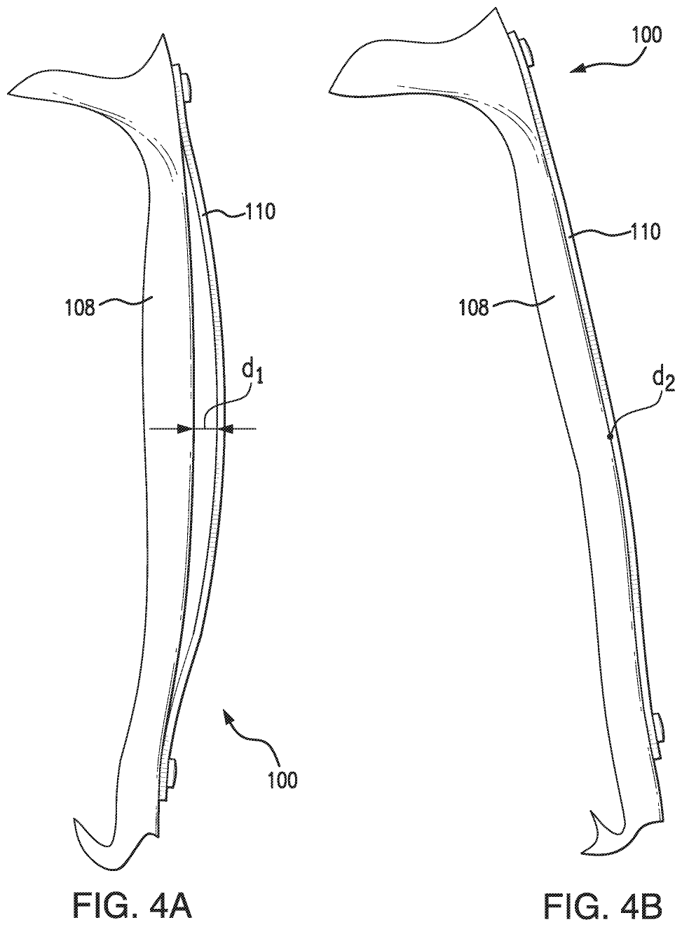

FIGS. 4A and 4B illustrate an exemplary spacing between the first leaf spring 108 and the second leaf spring 110. FIG. 4A depicts a first state in which an external load is not applied to the system. In the first state, the space between the first leaf spring 108 and the second leaf spring 110 may have a lateral distance d₁. In some embodiments, d₁ may be between 1 mm and 50 mm, 3 mm and 20 mm, or 5 mm and 10 mm. In some embodiments, d₁ may be approximately 6 mm. FIG. 4B depicts an exemplary second state in which an external load is applied to the system. This external load may be applied, for example, when a user walks, runs, climbs stairs, or otherwise applies a load to the system 100. As shown in FIG. 4B, when a force is applied in a dorsiflexion direction, a lateral distance d₂ of the space between the first leaf spring and the second leaf spring 110 may be reduced relative to lateral distance d₁. In the case illustrated in FIG. 4B, a force has been applied until the first leaf spring 108 contacts the second leaf spring 110, such that d₂ is zero. In other cases, d₂ may be a non-zero value less than d₁. In some embodiments, the first leaf spring may have a radius of curvature that is greater than a radius of curvature of the second leaf spring. In some embodiments, the radius of curvature of the first leaf spring may be greater than the radius of curvature of the second leaf spring when the system is in the first state. When the system is in the second state, the radius of curvature of the two leaf springs may be substantially the same, and/or a difference between the radii of curvature of the two leaf springs may be reduced relative to the difference between the radii of curvature of the two leaf springs when the system is in the first state.

For some users, it may be beneficial to provide a system that provides relatively less resistance to dorsiflexion up to a predetermined value of dorsiflexion, and greater resistance beyond that predetermined value. For example, during a normal gait, an ankle may flex in a late stance phase of a step up to a certain value, before the foot is lifted and the dorsiflexion is released. Flexion up to this value may be needed to permit an optimal gate, whereas flexion beyond this value may be undesirable and may potentially lead to injury or impede rehabilitation.

In some embodiments, the system 100 may have a first loading state in which the system has a first resistance to dorsiflexion, and a second loading state in which the system has a second resistance to dorsiflexion that is greater than the first resistance to dorsiflexion. As used herein, the term resistance to dorsiflexion does not refer to a resistive force itself, but instead refers to a tendency of the system to generate resistive force in response to an applied degree of dorsiflexion. In this sense, a resistance to dorsiflexion of the system is akin to a spring constant or elastic modulus of the system. In some embodiments, the system 100 may transition from the first loading state to the second loading state when a predetermined dorsiflexion value is exceeded. In some embodiments, the predetermined dorsiflexion value may be between 2° and 30°, 4° and 20°, 6° and 15°. In some embodiments, the predetermined dorsiflexion value may be approximately 12°.

In some embodiments, the transition from the first loading state to the second loading state may occur due to an interaction between the first leaf spring 108 and the second leaf spring 110. In embodiments in which a spacer is not disposed between the first leaf spring 108 and the second leaf spring 110, the system may deform with relatively less resistance until the space 114 between the first leaf spring 108 and the second leaf spring 112 is closed, and the two leaf springs contact one-another. Beyond this point, the system 100 may present substantially higher resistance to further dorsiflexion. For example, a slope of a stress-strain curve of the system may increase when the lateral distance between the first leaf spring 108 and the second leaf spring 110 reaches zero.

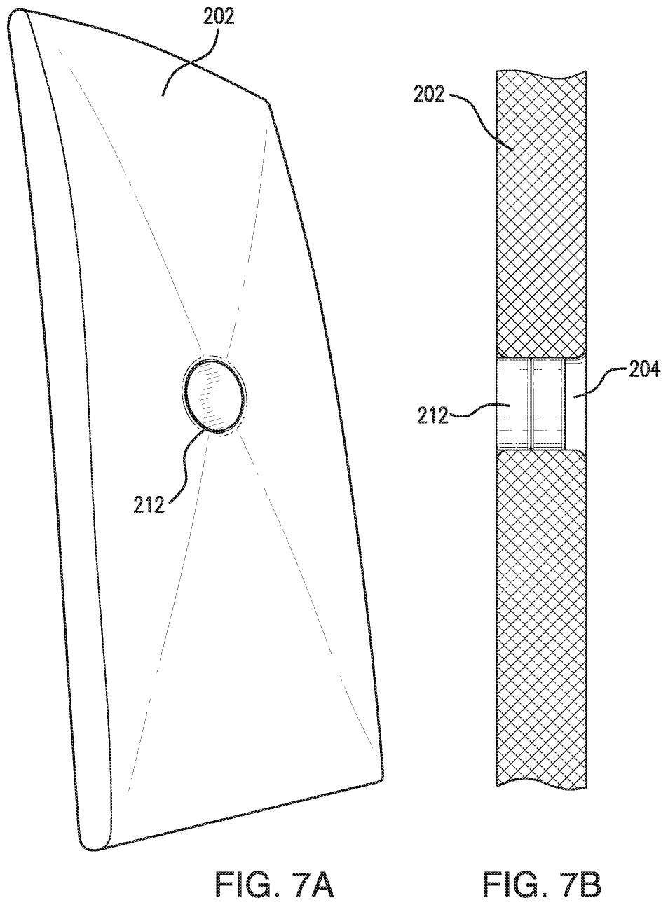

In embodiments in which one or more spacers 112 are disposed between the first leaf spring 108 and the second leaf spring 110, a dorsiflexion resistance profile of the system 100 may be governed in part by compression characteristics of the one or more spacers 112. In some embodiments, a spacer may have a first compressing state in which the spacer has a first resistance to compression, and a second compressing state in which the spacer has a second resistance to compression that is greater than the first resistance to compression. In some embodiments, one or more spacers may comprise a foam material. In some embodiments, the foam material may have a relatively lower resistance to compression in a first state, such as during compression or expulsion of air disposed within cells of the foam. In some embodiments, the foam material may have a relatively higher resistance to compression in a second state, such as during compression of the cell walls against one another, after air has largely been compressed or expelled from the foam. In other embodiments, one or more spacers may comprise elastomeric materials. One such embodiment is discussed with reference to FIG. 7 below. Other suitable materials may likewise be used to achieve a desired resistance profile.

In some embodiments, the system 100 may transition from the first loading state (e.g., lower resistance to dorsiflexion) to the second loading state (e.g., higher resistance to dorsiflexion) when one or more spacers 112 disposed between the first leaf spring 108 and the second leaf spring 110 transition from the first compressing state to the second compressing state. For example, a spacer 112 may transition from the first compressing state to the second compressing state when the system 100 reaches a predetermined dorsiflexion value, which may be between 2° and 30°, 4° and 20°, 6° and 15°. In some embodiments, the predetermined dorsiflexion value may be approximately 12°.

In some embodiments, a plurality of spacers having different compression characteristics may be provided. In some embodiments, placing different spacers between the first leaf spring 108 and the second leaf spring 110 may provide the system 100 with different dorsiflexion resistance profiles. For example, a resistance to dorsiflexion may be higher or lower in one or both of the first loading state or the second loading state, depending on the selection of the spacer. Additionally or alternatively, the transition from the first loading state to the second loading state may occur at a higher or lower predetermined dorsiflexion value, depending on the selection of the spacer. For example, the predetermined dorsiflexion value at which the system 100 transitions from the first loading state to the second loading state may be approximately 12° when a first spacer is used, and approximately 8° when a second spacer having different compression characteristics is used.

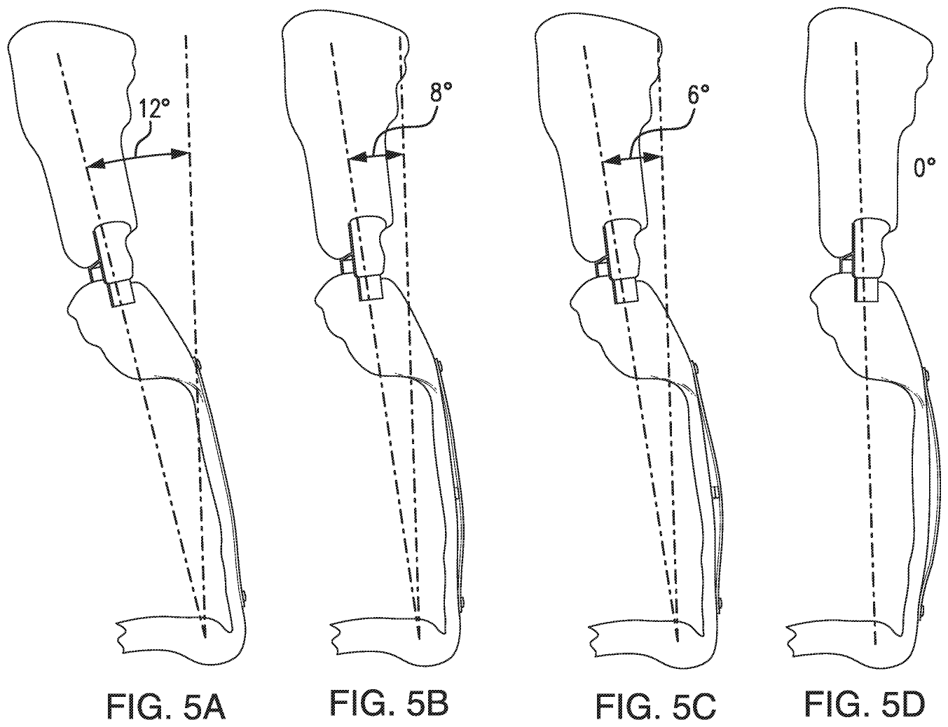

FIGS. 5A-5D provide exemplary illustrations of a system which permits variable dorsiflexion resistance profiles. In FIG. 5D, system 100 is shown in a state with no external load applied and a dorsiflexion measurement of 0°. In each of FIGS. 5A, 5B, and 5C, a load of 4 kg is applied at a position 70 cm from the ankle in a dorsiflexion direction. As illustrated, although an identical load is applied, the measured dorsiflexion differs depending upon the presence of one or more spacers between the first leaf spring and the second leaf spring. For example, in FIG. 5A, no spacers are present, and the exemplary system exhibits 12° of dorsiflexion under the 4 kg load. In FIG. 5B, one spacer is present, and the exemplary system exhibits 8° of dorsiflexion under the 4 kg load. In FIG. 5C, two spacers are present, and the exemplary system exhibits 6° of dorsiflexion under the 4 kg load. These are merely exemplary arrangements, and the number, geometry, and material properties of the spacers may be varied to achieve a desired relationship between applied load and dorsiflexion.

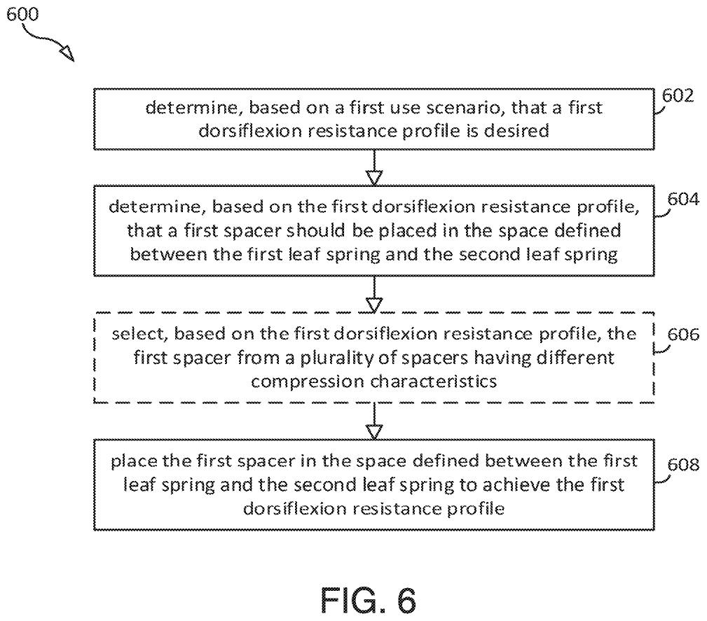

FIG. 6 depicts an exemplary method 600 for supporting a user's limb. In some embodiments, the system may be performed using any of the systems described herein, or any other suitable system. For example, the method may be performed using a system including one or more of: (i) a foot plate configured to engage the user's foot, (ii) a coupling configured to engage a portion of the user's leg, (iii) a first leaf spring disposed between the foot plate and the coupling, and/or (iv) a second leaf spring extending alongside at least a portion of the first leaf spring. In some embodiments, a space may be defined between the first leaf spring and the second leaf spring.

In step 602, it may be determined, based on a first use scenario, that a first dorsiflexion resistance profile of the system is desired. A use scenario may include any number of relevant considerations. For example, a use scenario may be as simple as specifying that the system is to be used for one or more of walking, running, climbing stairs, and/or playing certain sports. In other cases, a use scenario might contemplate characteristics of a user, such as their height, weight, activity levels, clinical factors, or rehabilitation progress. Based on these and any other relevant factors, a desired dorsiflexion resistance profile may be determined. The determined dorsiflexion resistance profile may have any of the characteristics described above. For example, it may be appropriate to select a higher resistance profile for a larger user, or it may be appropriate to select a resistance profile with a relatively low resistance in a first loading state but a high resistance in a second loading state for a user who desires relatively high mobility during normal use but wishes to guard against overextension.

In step 604, it may be determined, based on the first dorsiflexion resistance profile, that a first spacer should be placed in the space defined between the first leaf spring and the second leaf spring. For example, upon identifying a desired dorsiflexion resistance profile, one may determine whether a system provides the desired dorsiflexion resistance profile without using a spacer, or whether a spacer is needed to obtain the desired dorsiflexion resistance profile. In optional step 606, a first spacer may be selected, based on the first dorsiflexion resistance profile, from a plurality of spacers having different compression characteristics. In step 608, the first spacer may be placed in the space defined between the first leaf spring and the second leaf spring to achieve the first dorsiflexion resistance profile.

In some embodiments, the method 600 may further include determining, based on a second use scenario, that a second dorsiflexion resistance profile is desired. For example, the first use scenario may contemplate a user walking, and the second use scenario may contemplate the same user running or climbing stairs. In other exemplary embodiments, the first use scenario may contemplate the user walking relatively early in their rehabilitation, and the second use scenario may contemplate the user walking later in their rehabilitation. In some embodiments, the method 600 may further include selecting, based on the second dorsiflexion resistance profile, a second spacer having different compression characteristics than the first spacer, and placing the second spacer in the space defined between the first leaf spring and the second leaf spring. In some embodiments, the first spacer may be removed before the second spacer is placed between the first leaf spring and the second leaf spring. Third, fourth, fifth, etc. spacers may likewise be used to achieve third, fourth, fifth, etc. resistance profiles appropriate for respective use scenarios, as desired.

In some embodiments, multiple spacers may be placed within the space between the first leaf spring and the second leaf spring simultaneously. In some embodiments, providing multiple spacers may allow greater flexibility in controlling a resistance profile of the system. For example, the system may have multiple loading state transitions as a compression limit of each spacer is reached.

FIGS. 7A and 7B illustrate an exemplary multi-spacer embodiment. As illustrated, a first spacer 202 may be made from a first material having a first compressibility. In some embodiments, the first spacer 202 may define a cavity 204, which may be configured to receive one or more secondary spacers 212. In some embodiments, the one or more secondary spacers 212 may be made from a second material having a second compressibility that is different than the first compressibility. In some embodiments, the first spacer 202 may have a relatively high compressibility, and the one or more secondary spacers 212 may have a relatively low compressibility. In some embodiments, the first spacer 202 may have a lateral length that is greater than a lateral length of the one or more secondary spacers 212, either alone or in combination. For example, the first spacer 202 may be made from an elastomeric, foam, or otherwise resilient material, and may be sized to substantially fill the lateral distance between a first leaf spring and a second leaf spring of the system 100. The secondary spacers 212 may be made from a substantially nondeformable material, such as a metal or plastic, and may have a lateral distance that is less than the lateral distanced d₁ of the space in a state in which no load is applied.

In some embodiments, the system 100 may be in a first loading state while first spacer 202 is being deformed, and may transition to a second loading state when the lateral distance is reduced to engage the one or more secondary spacers 212. In some embodiments, the length of the one or more secondary spacers 212 may be varied to determine the point at which the system transitions from the first loading state to the second loading state. In some embodiments, smaller secondary spacers 212 may be positioned in series within the cavity 204 such that a combined length of the secondary spacers 212 may be varied by adding or subtracting one or more secondary spacers 212.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

1. A system for supporting a user's limb, the system comprising:
a foot plate configured to engage the user's foot;
a coupling configured to engage a portion of the user's leg;
a first leaf spring disposed between the foot plate and the coupling; and
a second leaf spring extending alongside at least a portion of the first leaf spring, such that a space is defined between the first leaf spring and the second leaf spring;
wherein in a first state in which an external load is not applied to the system, the space between the first leaf spring and the second leaf spring has a lateral distance d₁;
in a second state in which an external load is applied to the system, the space between the first leaf spring and the second leaf spring has a lateral distance d₂, d₂ being less than d₁; and
the space between the first leaf spring and the second leaf spring is configured to receive one or more spacers of a plurality of spacers, such that a dorsiflexion resistance profile of the system differs depending on how many and which of the one or more spacers is positioned within the space.

2. The system of claim 1, wherein the distance between the first leaf spring and the second leaf spring progressively decreases under increasing loads, and a slope of a stress-strain curve of the system increases when the distance reaches zero.

3. The system of claim 1, further comprising a spacer disposed in the space between the first leaf spring and the second leaf spring.

4. The system of claim 1, further comprising a plurality of spacers including at least a first spacer and a second spacer;
wherein the system has a first dorsiflexion resistance profile when only the first spacer is disposed in the space between the first leaf spring and the second leaf spring;
the system has a second dorsiflexion resistance profile when the second spacer is disposed, alone or in combination with the first spacer, in the space between the leaf spring and the second leaf spring; and
the second dorsiflexion resistance profile is different than the first dorsiflexion resistance profile.

5. The system of claim 4, wherein the first spacer has a first compression characteristic, and the second spacer has a second compression characteristic that is different than the first spacer.

6. The system of claim 4, wherein the first spacer is removable from the space between the first leaf spring and the second leaf spring by applying a load to the system in a plantarflexion direction.

7. The system of claim 4, wherein the first spacer has a first compressing state in which the spacer has a first resistance to compression, and a second compressing state in which the first spacer has a second resistance to compression that is greater than the first resistance to compression.

8. The system of claim 7, wherein the first spacer comprises a foam.

9. The system of claim 1, wherein the system has a first loading state in which the system has a first resistance to dorsiflexion, and a second loading state in which the system has a second resistance to dorsiflexion that is greater than the first resistance to dorsiflexion.

10. The system of claim 9, wherein the system transitions from the first loading state to the second loading state when a predetermined dorsiflexion value is exceeded, the predetermined dorsiflexion value being between 6° and 15°.

11. A method for supporting a user's limb, the method being performed using a system comprising (i) a foot plate configured to engage the user's foot, (ii) a coupling configured to engage a portion of the user's leg, (iii) a first leaf spring disposed between the foot plate and the coupling, and (iv) a second leaf spring extending alongside at least a portion of the first leaf spring, such that a space is defined between the first leaf spring and the second leaf spring, the method comprising:
determining, based on a first use scenario, that a first dorsiflexion resistance profile of the system is desired;
determining, based on the first dorsiflexion resistance profile, that a first spacer should be placed in the space defined between the first leaf spring and the second leaf spring;
placing the first spacer in the space defined between the first leaf spring and the second leaf spring to achieve the first dorsiflexion resistance profile.

12. The method of claim 11, further comprising selecting, based on the first dorsiflexion resistance profile, the first spacer from a plurality of spacers having different compression characteristics.

13. The method of claim 12, determining, based on a second use scenario, that a second dorsiflexion resistance profile is desired;
selecting, based on the second dorsiflexion resistance profile, a second spacer having different compression characteristics than the first spacer;
placing the second spacer in the space defined between the first leaf spring and the second leaf spring.

14. The method of claim 13, further comprising removing the first spacer from the space defined between the first leaf spring and the second leaf spring.

15. The method of claim 11, wherein the first spacer is a resilient member.

16. The method of claim 15, wherein the first spacer has a first compressing state in which the spacer has a first resistance to compression, and a second compressing state in which the first spacer has a second resistance to compression that is greater than the first resistance to compression.

17. The method of claim 15, wherein the first spacer comprises a foam.

18. The method of claim 11, further comprising placing a plurality of spacers in the space between the first leaf spring and the second leaf spring.

19. The method of claim 11, wherein the first dorsiflexion resistance profile comprises a first loading state in which the system has a first resistance to dorsiflexion, and a second loading state in which the system has a second resistance to dorsiflexion that is greater than the first resistance to dorsiflexion.

20. The method of claim 19, wherein the first dorsiflexion resistance profile comprises a transition from the first loading state to the second loading state at a predetermined dorsiflexion value, the predetermined dorsiflexion value being between 6° and 15°.

21. The method of claim 14, wherein the step of removing the first spacer from the space defined between the first leaf spring and the second leaf spring comprises applying a load to the system in a plantarflexion direction.
